(19) United States
(12) Patent Application Publication
(10) Pub. No.: US 2023/0320978 A1
(43) Pub. Date: Oct. 12, 2023

(54) COMPOSITIONS AND METHODS FOR LOWERING INTRACRANIAL PRESSURE

(71) Applicant: THE REGENTS OF THE UNIVERSITY OF COLORADO, A BODY CORPORATE, Denver, CO (US)

(72) Inventor: Prem SUBRAMANIAN, Denver, CO (US)

(21) Appl. No.: 18/133,784

(22) Filed: Apr. 12, 2023

Related U.S. Application Data
(63) Continuation of application No. PCT/US2021/050954, filed on Sep. 17, 2021.
(60) Provisional application No. 63/090,607, filed on Oct. 12, 2020.

(51) Int. Cl. A61K 9/00; A61K 31/5575; A61K 9/08; A61K 47/20; A61P 43/00

(52) U.S. Cl. CPC A61K 9/0043; A61K 31/5575; A61P 43/00; A61K 47/20; A61K 9/08

(57) ABSTRACT

Embodiments disclosed herein include nasal formulations and methods of use. In some embodiments, nasal formulations disclosed herein can include at least one agent comprising latanoprost, or pharmaceutically acceptable salt thereof; at least one adherence agent for prolonging nasal mucosal interaction of the formulation; and optionally, at least one pharmaceutically acceptable carrier or diluent. In some embodiments, methods include, but are not limited to, methods of increasing cerebrospinal fluid (CSF) outflow and reducing intracranial pressure (ICP) in subjects in need thereof.

### PRIORITY

This application is a Continuation Application of International Application No. PCT/US2021/050954, filed on Sep. 17, 2021, which claims the benefit of priority pursuant to 35 U.S.C. § 119(e) to U.S. Provisional Application No. 63/090,607, filed on Oct. 12, 2020. These applications are incorporated herein by reference in their entirety for all purposes.

### FIELD

Embodiments of the instant disclosure relate to novel compositions, methods and systems for reducing intracranial pressure (ICP) and/or increasing cerebral spinal fluid (CSF) outflow in a subject.

### BACKGROUND OF THE INVENTION

Cerebrospinal fluid (CSF) is produced in the lateral ventricles by active transport across the cell membranes of epithelial cells lining the arachnoid villi and is dependent upon Na/K ion channel activity. In normal subjects and subjects with disorders that elevate intracranial pressure (ICP) without physical obstruction within the ventricular system, the CSF flows freely through the ventricles, fills a number of cisterns as well as sulci along the surface of the brain, and is maintained in homeostasis via reabsorption through several putative pathways. Arachnoid granulations are outpouchings of the arachnoid mater into the dural venous sinuses that allow passage of CSF from the arachnoid space into the venous blood in a pressure-dependent manner; in most cases, CSF pressure exceeds venous sinus pressure, and this drives flow. CSF is also present in the meningeal coverings of the cranial nerves that exist before they exit the cranial compartment, and CSF can flow along the most proximal portion of spinal root ganglia and nerves. Normal CSF outflow occurs along these nerves and nerve roots. Defects in CSF outflow can lead to elevated intracranial pressure (ICP). In some cases, elevated ICP leads to optic nerve swelling and vision loss.

No medications are currently available that increase CSF outflow. Current clinical methods of increasing CSF outflow include CSF diversion by surgical means (shunting from the ventricles into the thorax or abdomen) as the primary option. Therefore, there is a need for identification of medicinal regimens for increasing CSF outflow as a means of reducing intracranial pressure in a subject.

### SUMMARY

Embodiments of the instant disclosure relate to novel compositions, methods and systems for reducing intracranial pressure (ICP) and/or increasing cerebral spinal fluid (CSF) outflow in a subject. In certain embodiments, nasal formulations including, but not limited to, latanoprost, or pharmaceutically acceptable salt thereof or combination agents thereof are disclosed. In other embodiments, nasal formulations disclosed herein can include at least one adherence agent for prolonging nasal mucosal interaction of the nasal formulation containing latanoprost. In certain embodiments, nasal formulations disclosed herein can include, but are not limited to, latanoprost and at least one adherence agent. In some embodiments, methods of using nasal formulations disclosed herein for increasing CSF outflow and/or reducing ICP in a subject are provided. In certain embodiments compositions disclosed herein can include another prostaglandin F2 alpha agonist alone or in combination with latanoprost or derivative thereof.

In certain embodiments, a subject experiencing optic disc swelling, choroidal engorgement, positional headache, pulse-synchronous tinnitus, cerebral venous sinus stenosis, refractive shifts, other symptoms of ICP or a combination thereof can be treated by nasal formulations disclosed herein. In some embodiments, a subject can be experiencing an ICP-related condition or a combination of ICP-related conditions. In accordance with these embodiments, a subject can have one or more of Spaceflight-Associated Neuro-Ocular Syndrome, idiopathic intracranial hypertension, and pseudotumor cerebri syndrome. It is contemplated herein that the compositions and methods disclosed herein can be used to treat, reduce onset of and/or prevent any ICP-related condition. In some embodiments, symptoms alleviated by these compositions can include, but are not limited to, reducing cranial pressure and/or increasing CSF outflow.

In certain embodiments, methods disclosed herein can include treating a subject having an ICP-related condition by administering a latanoprost or other prostaglandin F2 alpha agonist (e.g. bimatoprost and/or tafluprost and/or vyzulta) or latanoprost combination formulation with another standard agent to the subject intranasally, by topical (e.g. within the nasal cavity), by spray, by mist, by drops, or by aerosol delivery or any other intranasal delivery method. In accordance with these embodiments, nasal formulations delivery devices disclosed herein can include droppers, by aerosol or other nasal delivery device. In accordance with these embodiments, delivery of nasal formulations disclosed herein can be by drop, mist or aerosol, ointment, other nasal delivery method or by timed-delivery method.

In certain embodiments, one or more adherence agent or thickening agent can be added to the nasal formulation in order to enhance and/or prolong mucosal membrane contact of the formulations disclosed herein. In accordance with these embodiments, an adherence agent can be a carrier or diluent having at least one of increased density, increased stickiness or having gelatinous properties. In accordance with these embodiments, an adherence agent can be a carrier or diluent including, but not limited to, solid carriers or diluents, liquid carriers or diluents, gelatinous carriers or diluents, polymer-containing carriers or diluents or a combination thereof. In some embodiments, the at least one adherence agent can include cellulose or a derivative thereof, a starch, a wax, a gel, a synthetic polymer, a natural polymer, or combination thereof. In certain embodiments, the carrier or diluent can be liquid carrier or diluent including at least one of water, propylene glycol, PBS and pharmaceutically acceptable alcohols in combination with at least one adherence agent.

In certain embodiments, nasal formulations disclosed herein can include latanoprost or a latanoprost derivative thereof or other prostaglandin F2 alpha agonist (e.g. bimatoprost or tafluprost or vyzulta) having the same activities as latanoprost such as a conjugate or other derivative. In certain embodiments, any commercially available form of latanoprost can be administered alone or in combination with other agents to treat a condition disclosed herein (latanoprost: e.g. Xalatan™, Xelpros™, Monoprost™ or others). In accordance with these embodiments, latanoprost or other prostaglandin F2 alpha agonist (e.g. bimatoprost or tafluprost or vyzulta) can be administered at a concentration of about 0.01 mg/ml to about 20.0 mg/ml, or about 0.05 mg/ml to about 10.0 mg/ml, or about 0.1 mg/ml to about 5.0 mg/ml to a subject or about 0.05 mg/ml to about 2.0 mg/ml. In some embodiments, nasal formulations disclosed herein can include a viscous liquid of latanoprost, semi-solid particles of latanoprost, micelles containing latanoprost, slow-release particles, coated microparticles containing latanoprost or the like or other prostaglandin F2 alpha agonist (e.g. bimatoprost and/or tafluprost and/or vyzulta) or a combination thereof in order to improve adherence and outcome. In other embodiments, formulations disclosed herein have higher viscosities than eye formulations, at least 1.5 times, 2 times, 3 times or higher viscosity that eye formulations.

In certain embodiments, nasal formulations disclosed herein can be a solution, a suspension, or an emulsion. In accordance with these embodiments, nasal formulations disclosed herein can include, but are not limited to, polymers of carbopol, chitosan, sodium carboxymethyl cellulose (NaCMC), hydroxypropyl methylcellulose (HPMC), hydroxypropyl cellulose methylcellulose, poloxamer, polyoxyethylene, pluronic-poly(acrylic acid) copolymer, carbomer, chitosan, polyvinyl alcohol (PVA), poly(N-isopropylacrylamide) (PNiPAAm), methocel A4M, polymethacrylic acid and polyethylene glycol (P(MAA-g-EG), polyvinylacetal diethylamino acetate, or a combination thereof.

In certain embodiments, the present disclosure provides for methods of increasing CSF outflow and/or decreasing ICP in a subject in need thereof. In some embodiments, methods of increasing CSF outflow and/or decreasing ICP in a subject in need thereof can include administering nasal formulations disclosed herein. In accordance with these embodiments, methods of increasing CSF outflow can include administering a nasal formulation that includes, but may not be limited to, latanoprost or a pharmaceutically acceptable salt thereof or a derivative thereof. In certain embodiments, latanoprost or a pharmaceutically acceptable salt thereof or a derivative thereof can be administered to a subject at a concentration of about 0.01 mg/ml to about 20.0 mg/ml. In some embodiments, nasal formulations disclosed herein can include administering formulations, daily, twice daily or more; or every other day, or as needed to relieve ICP and/or increase CSF outflow or other similar condition.

In certain embodiments, the present disclosure provides for methods of reducing ICP in a subject. In accordance with these embodiments, methods for administering nasal formulation disclosed herein can be used to treat a subject having one or more of, optic disc swelling, choroidal engorgement, positional headache, pulse-synchronous tinnitus, cerebral venous sinus stenosis, refractive shifts, or a combination thereof. In some embodiments, a subject can have one or more ICP complications. In accordance with these embodiments, a subject can have one or more of Spaceflight-Associated Neuro-Ocular Syndrome, idiopathic intracranial hypertension, and pseudotumor cerebri syndrome. In some embodiments, methods for treating these conditions or complications of ICP can include, but is not limited to, administering a latanoprost-containing nasal formulation or other prostaglandin F2 alpha agonist (e.g. bimatoprost and/or tafluprost and/or vyzulta) to the subject by intranasal delivery, by topical delivery to the nasal passage, spray, mist, drop, and/or aerosol delivery. In some embodiments, methods for treating these conditions or complications of ICP can include, but is not limited to, administering a latanoprost-containing nasal formulation to the subject at least once a day by intranasal delivery, by topical delivery to the nasal passage, spray, mist, drop, and/or aerosol delivery.

In certain embodiments, the present disclosure provides for kits including, but not limited to, a nasal formulation containing latanoprost or salt derivative thereof or other derivative thereof, as disclosed herein and at least one container.

### BRIEF DESCRIPTION OF THE DRAWINGS

FIGS. 1A and 1B represent exemplary experiments of the instant disclosure, illustrating the presence of fluorescent tracer (FIG. 1A) or absence thereof (FIG. 1B) in a ventricle of a mouse brain in accordance with certain embodiments of the present disclosure.

FIG. 2 represents an exemplary experiment of the instant disclosure, illustrating representative tracer recovery in the presence of prostaglandin F2-alpha analogues relative to controls in accordance with certain embodiments of the present disclosure.

FIG. 3 represents an exemplary experiment of the instant disclosure, illustrating representative tracer recovery in the presence of prostaglandin F2-alpha analogues relative to controls in accordance with certain embodiments of the present disclosure.

FIG. 4 represents an exemplary experiment of the instant disclosure, illustrating representative tracer recovery in the presence of prostaglandin F2-alpha analogues in accordance with certain embodiments of the present disclosure.

FIG. 5 represents an exemplary experiment of the instant disclosure, illustrating representative tracer recovery in the presence of prostaglandin F2-alpha analogues in accordance with certain embodiments of the present disclosure.

### DEFINITIONS

Terms, unless defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” are used interchangeably herein and refer to any mammalian subject for whom diagnosis, treatment, prophylaxis or therapy is desired, particularly humans.

As used herein, “treat,” “treating” or “treatment” can refer to treating, reversing, ameliorating, or inhibiting onset or inhibiting progression of a health condition or disease or a symptom of the health condition or disease.

The term “cerebrospinal fluid” or “CSF” can refer to a clear, colorless body fluid that can serve as a mechanical and immunological protection of the brain inside the skull. As applied to a subject, CSF pressures can vary by about 1 millimeter of mercury (mmHg) in adults through shifts in production and absorption of CSF.

The term “intracranial pressure” or “ICP” can refer to the pressure inside the skull and in the brain tissue and can be due to CSF. As applied to a subject, ICP can be measured in mmHg and, at rest, is typically about 7-15 mmHg for a human adult subject, positioned surpine.

The term “Spaceflight-Associated Neuro-Ocular Syndrome” or “SANS” can refer to a condition that occurs when a microgravity environment causes cephalad fluid shifts in astronauts. As applied to a subject herein, ICP changes can occur, and if left untreated, SANS can lead to vision alterations and potentially other deleterious health effects.

Unless otherwise defined, all technical terms used herein have the same meaning as commonly understood by one of ordinary skill in the art to which this disclosure belongs.

### DETAILED DESCRIPTION OF THE INVENTION

The following sections, certain exemplary compositions and methods are described in order to detail certain embodiments of the disclosure. It will be obvious to one skilled in the art that practicing the certain embodiments does not require the employment of all or even some of the specific details outlined herein, but rather that concentrations, times and other specific details can be modified through routine experimentation. In certain situations, well known methods, or components have not been included in the description.

Embodiments of the instant disclosure relate to novel compositions, methods, and kits including nasal formulations containing at least latanoprost, or other prostaglandin F2 alpha agonist (e.g. bimatoprost and/or tafluprost and/or vyzulta), a derivative thereof or conjugate thereof, or a pharmaceutically acceptable salt thereof. Other embodiments of the instant disclosure relate to methods of administering nasal formulations disclosed herein for reducing ICP, increasing CSF outflow, or a combination thereof.

In certain embodiments, nasal formulations can include, but are not limited to, latanoprost or other prostaglandin F2 alpha agonist (e.g. bimatoprost and/or tafluprost and/or vyzulta), or a pharmaceutically acceptable salt thereof or derivative thereof having the same or similar activity of latanoprost. Latanoprost is an analog of prostaglandin F2a that can act as a selective agonist at the prostaglandin F receptor. Latanoprost is also known by a chemical name of isopropyl (Z)-7-[(1R,2R,3R,5S)-3,5-dihydroxy-2-[(3R)3-hydroxy-5-phenylpentyl]-cyclopentyl] hept-5-enoate.

In some embodiments, latanoprost used in the formulations disclosed herein can be in the form of an ester prodrug. The term “ester” herein can refer a compound which is produced by modifying a functional group (e.g. hydroxyl, carboxyl, amino or the like group). Examples of the “ester” include “esters formed with a hydroxyl group” and “esters formed with a carboxyl group.” The term “ester” can mean an ester whose ester residue is a “conventional protecting group” or a “protecting group removable in vivo by a biological method such as hydrolysis”. In some embodiments, the term “conventional protecting group” can mean a protecting group removable by a chemical method such as hydrogenolysis, hydrolysis, electrolysis or photolysis. In some embodiments, the term “protecting group removable in vivo by a biological method such as hydrolysis” can mean a protecting group removable in vivo by a biological method such as hydrolysis to produce a free acid or its salt.

In some embodiments, latanoprost or other prostaglandin F2 alpha agonist (e.g. bimatoprost and/or tafluprost and/or vyzulta) used in the formulations herein can be in the form of a pharmaceutically acceptable salt. By “salt” or “pharmaceutically acceptable salt”, it is meant those salts which are, within the scope of sound medical judgment, suitable for use in contact with the tissues of humans and lower animals without undue toxicity, irritation, and allergic response, commensurate with a reasonable benefit to risk ratio, and effective for their intended use. A “pharmacologically acceptable salt” can refer to a salt, which can be formed when latanoprost or other prostaglandin F2 alpha agonist (e.g. bimatoprost and/or tafluprost and/or vyzulta) has an acidic group such as carboxyl or a basic group such as amino or imino. In some embodiments, a latanoprost salt formed with an acidic group herein can include alkali metal salts such as a sodium salt, potassium salt or lithium salt, alkaline earth metal salts such as a calcium salt or magnesium salt, metal salts such as an aluminum salt or iron salt; amine salts, e.g., inorganic salts such as an ammonium salt and organic salts such as a t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt or tris(hydroxymethyl)aminomethane salt; and amino acid salts such as a glycine salt, lysine salt, arginine salt, ornithine salt, glutamate or aspartate. In some embodiments, a latanoprost or other prostaglandin F2 alpha agonist (e.g. bimatoprost and/or tafluprost and/or vyzulta) salt formed with a basic group herein can include hydro-halides such as a hydrofluoride, hydrochloride, hydrobromide or hydroiodide, inorganic acid salts such as a nitrate, perchlorate, sulfate or phosphate; lower alkanesulfonates such as a methanesulfonate, trifluoromethanesulfonate or ethanesulfonate, arylsulfonates such as a benzenesulfonate or p-toluenesulfonate, organic acid salts such as an acetate, malate, fumarate, succinate, citrate, ascorbate, tartrate, oxalate or maleate; and amino acid salts such as a glycine salt, lysine salt, arginine salt, ornithine salt, glutamate or aspartate. In certain embodiments, when a pharmacologically acceptable salt of latanoprost remains in the atmosphere or is recrystallized, it can absorb water to form a hydrate of use in formulations disclosed herein.

In some embodiments, latanoprost or other prostaglandin F2 alpha agonist (e.g. bimatoprost and/or tafluprost and/or vyzulta) used in formulations disclosed herein can be in the form of another latanoprost derivative. In some embodiments, the term “other derivative” can mean a derivative of latanoprost other than the above-described “ester” or the above-described “pharmacologically acceptable salt” which can be formed, if it has an amino and/or carboxyl group or other conjugate form or other active derivative thereof.

In some embodiments, nasal formulations disclosed herein can include latanoprost contained within or formed into particles. In some embodiments, particle forms of latanoprost can be a solid particle. In some embodiments, particle forms of latanoprost can be a semi-solid particle. In some embodiments, a particle of latanoprost can be prepared by those of skill in the art using known methods for such preparation. Non-limiting examples of methods of preparing latanoprost particles can include spray-drying, spray-freeze drying, lyophilization, evaporation, micronization, nanosization, crystallization, and/or other known methods.

In some embodiments, nasal formulations can contain latanoprost or other prostaglandin F2 alpha agonist (e.g. bimatoprost and/or tafluprost and/or vyzulta) in the form of a particle. In accordance with these embodiments, latanoprost-containing particles can be about 100, 75, 50, 25, 20, 15, 10, 9.5, 9.0, 8.5, 8.0, 7.5, 7.0, 6.5, 6.0, 5.5, 5.0, 4.5, 4.0 microns or smaller in size. In some embodiments, nasal formulations can contain latanoprost in the form of a particle where greater than about 90% or about 100% of the latanoprost particles can have a particle size less than about 15 microns (e.g., about 0.5, 1, 2, 3, 4, 5, 6, 7, 8, 9, 10, 11, 12, 13, 14, 15). In some embodiments, particles containing latanoprost can be part of a formulation for nasal delivery and mucosal adhesion with enhance effects for treatment of conditions disclosed herein.

In some embodiments, nasal formulations disclosed herein can include a concentration of about 0.01 mg/ml to about 20 mg/ml latanoprost or other prostaglandin F2 alpha agonist (e.g. bimatoprost and/or tafluprost and/or vyzulta). In some embodiments, nasal formulations disclosed herein can include a concentration of about 0.01 mg/ml, about 0.1 mg/ml, about 0.2 mg/ml, about 0.3 mg/ml, about 0.4 mg/ml, about 0.5 mg/ml, about 1.0 mg/ml, about 2.5 mg/ml, about 5 mg/ml, about 7.5 mg/ml, about 10 mg/ml, or about 20 mg/ml latanoprost alone or other prostaglandin F2 alpha agonist (e.g. bimatoprost and/or tafluprost and/or vyzulta) or in combination with a standard agent to treat conditions disclosed herein.

In some embodiments, nasal formulations disclosed herein can be a solution, a suspension, a paste, a gel or an emulsion. In some embodiments, nasal formulations disclosed herein can include at least one pharmaceutically acceptable carrier or diluent. In some embodiments, pharmaceutically acceptable carriers and diluents suitable for use herein can be selected from solid carriers or diluents, liquid carriers or diluents, gel carriers or diluents or a combination thereof.

In certain embodiments, nasal formulations disclosed herein can be a solution, a suspension, a paste, a cream, or an emulsion. In accordance with these embodiments, nasal formulations disclosed herein can include, but are not limited to, polymers of carbopol, chitosan, sodium carboxymethyl cellulose (NaCMC), hydroxypropyl methylcellulose (HPMC), hydroxypropyl cellulose methylcellulose, poloxamer, polyoxyethylene, pluronic-poly(acrylic acid) copolymer, carbomer, chitosan, polyvinyl alcohol (PVA), poly(N-isopropylacrylamide) (PNiPAAm), methocel A4M, polymethacrylic acid and polyethylene glycol (P(MAA-g-EG), polyvinylacetal diethylamino acetate, surfactants, high molecular weight surfactants, poloxamers (e.g. poloxamer 407 or 403 etc.), solvents or other agents of use to dissolve or maintain agents disclosed herein, DMSO (dimethyl sulfoxide) or equivalent agent known in the art or a combination thereof.

In certain embodiments, a carrier or diluent can be a liquid carrier or diluent comprising at least one of water, propylene glycol and pharmaceutically acceptable alcohols. Pharmacologically suitable fluids for use herein can include, but are not limited to, polar solvents, including, but not limited to, compounds that contain hydroxyl groups or other polar groups. Solvents for use herein can include, but are not limited to, water or alcohols, such as ethanol, isopropanol, and glycols including propylene glycol, polyethylene glycol, polypropylene glycol, glycol ether, glycerol and polyoxyethylene alcohols. Polar solvents can also include protic solvents, including, but not limited to, water, aqueous saline solutions with one or more pharmaceutically acceptable salt(s), alcohols, glycols or a mixture there of. In some embodiments, water for use in the present formulations can meet or exceed the applicable regulatory requirements for use in inhaled drugs.

In some embodiments, a carrier or diluent can be a gel carrier or diluent or a combination thereof. In some embodiments, the latanoprost or other prostaglandin F2 alpha agonist (e.g. bimatoprost and/or tafluprost and/or vyzulta) can be a viscous liquid. In certain embodiments, nasal formulations herein can include at least one viscosity and/or density enhancing agent. Examples of viscosity and/or density enhancing agents that can be added include, but are not limited to, carboxymethylcellulose (CMC), veegum, tragacanth, bentonite, hydroxypropylmethylcellulose, hydroxypropylcellulose, hydroxyethylcellulose, poloxamers (e.g. poloxamer 407), polyethylene glycols, alginates xanthum gums, carageenans and carbopols. In some embodiments, a viscosity enhancing agent for use herein can possess thixotropic properties to ensure that the formulation can assume a gel-like appearance at rest, characterized by a high viscosity value. In some embodiments, if a nasal formulation disclosed herein is subjected to shear forces, such as those caused by agitation prior to spraying, the viscosity of the formulation can decrease transiently to such a level to enable it to flow readily through the spray device and exit as a fine mist spray. In some embodiments, a mist as disclosed herein can be capable of infiltrating the mucosal surfaces of the anterior regions of the nose (frontal nasal cavities), the frontal sinus, the maxillary sinuses and the turbinate which overlies the conchas of the nasal cavities. In some embodiments, once deposited in a subject, the viscosity of the nasal formulations disclosed herein can increase to a sufficient level to assume a gel-like form and remain in the nasal mucosa longer for improved treatment. In certain embodiments, nasal formulations herein can include a viscosity enhancing agent in an amount of about 0.1% (w/w) to about 5% (w/w), based on the total weight of the formulation. In certain embodiments, formulations disclosed herein can have about 1.1, to about 1.2, to about 1.3, to about 1.4, to about 1.5, to about 2.0, to about 3.0 or more times the viscosity of an eye formulation. In some embodiments, increased viscosity herein can increase adherence to nasal mucosa and increase persistence of exposure to the agents contemplated herein.

In some embodiments, the disclosed nasal formulations can include at least one adherence agent for prolonging nasal mucosal interaction of the formulation. In some embodiments, an adherence agent for use herein can be a cellulose or a derivative thereof, a starch, a wax, a gel, a synthetic polymer, a natural polymer, and the like. In some embodiments, the disclosed nasal formulations can include at least one polymer. In some embodiments, a polymer suitable for use in the formulations herein can be a mucoadhesive polymer. A “mucoadhesive polymer” as understood herein is a natural, or synthetic macromolecules capable of adhering to mucosal tissue surfaces. In some embodiments, a polymer suitable for use herein can be carbopol, chitosan, sodium carboxymethyl cellulose (NaCMC), hydroxypropyl methylcellulose (HPMC), hydroxypropyl cellulose methylcellulose, poloxamer, polyoxyethylene, pluronic-poly(acrylic acid) copolymer, carbomer, chitosan, polyvinyl alcohol (PVA), poly(N-isopropylacrylamide) (PNiPAAm), methocel A4M, polymethacrylic acid and polyethylene glycol (P(MAA-g-EG), polyvinylacetal diethylamino acetate, or a combination thereof. In some embodiments, nasal formulations herein can include a mucoadhesive polymer having a concentration of about 0.1% (w/w) to about 25% (w/w) or about 1.01% (w/w) to about 20% (w/w).

In some embodiments, nasal formulations disclosed herein can have a pH of about 2.0 to about 9.0; or about 4.0 to about 8.0; or about 5.5 to about 7.5 (e.g., about 2, 3, 4, 5, 6, 7, 8, 9). In some embodiments, nasal formulations herein can include a pH-stabilizing buffer. In some embodiments, a pH buffer herein can include any known pharmaceutically suitable buffer which are physiologically acceptable upon administration intranasally.

In some embodiments, nasal formulations disclosed herein can be free of pathogenic organisms (e.g., sterile). In some embodiments, the nasal composition can be formulated to be a pharmaceutical formulation. Processes which can be considered for achieving sterility in certain embodiments herein can include any appropriate sterilization steps known in the art. In some embodiments, latanoprost can be produced under sterile conditions. In some embodiments, latanoprost mixing and packaging can be conducted under sterile conditions. In some embodiments, nasal formulations disclosed herein can be sterile filtered and filled in vials, including unit dose vials providing sterile unit dose formulations which can be used in a nasal spray device. In some embodiments, each unit dose vial herein can be sterile and can be suitably administered without contaminating other vials or the next dose. In some embodiments, one or more ingredients in the nasal formulations herein can be sterilized by steam, gamma radiation or prepared using or mixing sterile steroidal powder and other sterile ingredients where appropriate. In some embodiments, nasal formulations herein can be prepared and/or handled under sterile conditions or can be sterilized before or after packaging.

In some embodiments, in addition to or in lieu of sterilization, nasal formulations disclosed herein can include a pharmaceutically acceptable preservative. Preservatives suitable for use herein can include, but are not limited to, those that protect the solution from contamination with pathogenic particles, including phenylethyl alcohol, benzalkonium chloride or benzoic acid, or benzoates such as sodium benzoate and phenylethyl alcohol. In some embodiments, a preservative for use in the present formulations can be benzalkonium chloride. In certain embodiments, formulations herein can have from about 0.001% to about 10.0% w/w of benzalkonium chloride, or from about 0.01% v/w phenylethyl alcohol. In certain embodiments, formulations herein can have preserving agents present in the formulations herein at an amount from about 0.001% to about 1% w/w.

In some embodiments, nasal formulations provided herein can include about 0.001% to about 90%, about 0.001% to about 50%, about 0.001% to about 25%, about 0.001% to about 10%, or about 0.001% to about 1% of one or more emulsifying agents, wetting agents, or suspending agents. Such agents for use herein can include, but are not limited to, polyoxyethylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-21actylate; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerised fatty acids of soya bean oil; oxidatively polymerised soya bean oil; and pectin extract. In certain embodiments herein, the present formulations may have polysorbate 80, microcrystalline cellulose, carboxymethylcellulose sodium and/or dextrose.

In some embodiments, nasal formulations provided herein can include about 0.001% to about 90%, about 0.001% to about 50%, about 0.001% to about 25%, about 0.001% to about 10%, or about 0.001% to about 1% of one or more pharmacologically suitable excipients and additives. Excipients and additives generally have no pharmacological activity, or at least no undesirable pharmacological activity. The concentration of excipients and additives herein can vary, although the presence or absence of these excipients and additives, or their concentration is not an essential feature of the formulations herein. Excipients and additives suitable for use herein can include, but are not limited to, surfactants, moisturizers, stabilizers, complexing agents, antioxidants, or other additives known in the art. Complexing agents include, but are not limited to, ethylenediaminetetraacetic acid (EDTA) or a salt thereof, such as the disodium salt, citric acid, nitrilotriacetic acid and the salts thereof. In some embodiments, nasal formulations herein can include a humectant. In some embodiments, nasal formulations herein can include from about 0.001% to about 5% (e.g., about 0.001%, 0.01%, 0.1%, 0.5%, 1%, 2%, 3%, 4%, 5%) by weight of a humectant to inhibit drying of the mucous membrane and/or to prevent irritation. Any of a variety of pharmaceutically acceptable humectants can be employed herein, including, but not limited to, sorbitol, propylene glycol, polyethylene glycol, glycerol or mixtures thereof, and the like.

In some embodiments, nasal formulations provided herein can include one or more solvents or co-solvents to increase the solubility of any of the components of the present formulation. In some embodiments, solvents in the formulations described herein can be about 0.001% to about 90%, about 0.001% to about 50%, about 0.001% to about 25%, about 0.001% to about 10%, or about 0.001% to about 10% of one or more solvents or co-solvents. Solvents or co-solvents for use herein include, but are not limited to, hydroxylated solvents or other pharmaceutically-acceptable polar solvents, such as alcohols including isopropyl alcohol, glycols such as propylene glycol, polyethylene glycol, polypropylene glycol, glycol ether, glycerol, and polyoxyethylene alcohols or for example, DMSO.

In some embodiments, nasal formulations provided herein can include at least one tonicity agent. Tonicity agents for use herein can include, but are not limited to sodium chloride, potassium chloride, zinc chloride, calcium chloride or mixtures thereof. Other osmotic adjusting agents can also include, but are not limited to, mannitol, glycerol, and dextrose or mixtures thereof. In some embodiments, nasal formulations herein can have about 0.01% to about 8% w/w, or about 1% to about 6% w/w total amount of tonicity agent(s).

In some embodiments, nasal formulations provided herein can be stable. In some embodiments, stability of formulations provided herein can refers to the length of time at a given temperature that greater than about 80%, 85%, 90% or 95% of the initial amount of drug substance, (e.g., latanoprost or other prostaglandin F2 alpha agonist (e.g. bimatoprost and/or tafluprost and/or vyzulta)) is present in the formulation. In some embodiments, nasal formulations provided herein can be stored from about 4° C. to about 50° C.; about 4° C. to about 40° C.; or about 4° C. to about 30° C.; or about °4 C. to about 35° C., for about a week, for about 2 weeks, for about 3 weeks, for about 1, 2, 3, 6, 12, 18, 24 months or about 36 months depending on stability. In some embodiments, nasal formulations disclosed herein can be suitable for administration to a subject in need thereof after storage of about 1 day, to 1 week, to 2 weeks, to about 3 weeks, to about 1, 2, 3, 6, 12, 18, 24 or 36 months at about 4° C. to about 30° C. In some examples, more than 80%, more than 85%, more than 90%, or more than 95% of the initial amount of drug substance (e.g., latanoprost) can remain after storage of the formulations for more than about 1, 2, 12, 18, 24 or 36 months between about 15° C. and about 30° C.

In certain embodiments, nasal formulations of the present disclosure can be manufactured in any conventional manner. In some embodiments, nasal formulations herein can be made by thoroughly mixing the ingredients described herein at ambient or elevated temperatures in order to achieve solubility of ingredients where appropriate. In some embodiments, preparation of latanoprost or other prostaglandin F2 alpha agonist (e.g. bimatoprost or tafluprost or vyzulta) having a particle size distribution profile disclosed herein can be obtained or generated by any conventional means known in the art, or by minor modification of such means. In some embodiments, suspensions of latanoprost particles herein can rapidly undergo particulate size reduction when subjected to “jet milling” (e.g., high pressure particle in liquid milling) techniques. Other known methods for reducing particle size into the micrometer range suitable for use herein can include, but are not limited to mechanical milling, the application of ultrasonic energy and/or the like.

In some embodiments, nasal formulations disclosed herein can incorporate lipid or fatty acid-based carriers, processing agents, or delivery vehicles, to provide improved formulations for mucosal delivery of latanoprost or other prostaglandin F2 alpha agonist (e.g. bimatoprost and tafluprost or other). In some embodiments, a variety of formulations and methods are provided for mucosal delivery herein which can include latanoprost or other prostaglandin F2 alpha agonist (e.g. bimatoprost and tafluprost or other), admixed or encapsulated by, or coordinately administered with, a liposome, mixed micellar carrier, or emulsion, to enhance chemical and physical stability and increase the half-life of the drug (e.g., by reducing susceptibility to proteolysis, chemical modification and/or denaturation) upon mucosal delivery. Like liposomes, unsaturated long chain fatty acids, which also have enhancing activity for mucosal absorption, can form closed vesicles with bilayer-like structures (so-called “ufasomes”). These can be formed, for example, using oleic acid to entrap biologically active peptides and proteins for mucosal, e.g., intranasal, delivery within this disclosure. In some embodiments, other delivery systems within this disclosure can combine the use of polymers and liposomes to ally the advantageous properties of both vehicles such as encapsulation inside the natural polymer fibrin.

In some embodiments, nasal formulations herein can include long and/or medium chain fatty acids, as well as surfactant mixed micelles with fatty acids. Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers. In some embodiments, nasal formulations herein can include long chain fatty acids, especially fusogenic lipids (e.g., unsaturated fatty acids and monoglycerides such as oleic acid, linoleic acid, linoleic acid, monoolein, etc.) can provide useful carriers to enhance mucosal delivery of insulin, analogs and mimetics, and other biologically active agents disclosed herein. Medium chain fatty acids (C6 to C12) and monoglycerides have also been shown to have enhancing activity in intestinal drug absorption and can be adapted for use within the mucosal delivery formulations and methods of this disclosure. In addition, sodium salts of medium and long chain fatty acids are effective delivery vehicles and absorption-enhancing agents for mucosal delivery of biologically active agents within this disclosure. In some embodiments, formulation herein can include one or more fatty acids. Fatty acids can be employed in soluble forms of sodium salts or by the addition of non-toxic surfactants, e.g., polyoxyethylated hydrogenated castor oil, sodium taurocholate, and the like. Other fatty acid and mixed micellar preparations that are within this disclosure can include, but are not limited to, Na caprylate (C8), Na caprate (C10), Na laurate (C12) or Na oleate (C18), and can be combined with bile salts, such as glycocholate and taurocholate.

In some embodiments, nasal formulations of the present disclosure can be formulated into a dosage form for pharmaceutical administration. Suitable dosage forms include, without limitation, liquids, ointments, creams, emulsions, lotions, gels, bioadhesive gels, sprays, aerosols, pastes, foams, sunscreens, capsules, microcapsules, suspensions, pessary, powder, semi-solid dosage form, and the like. In some embodiments, formulations herein can be formulated into a liquid dispersion, gel, aerosol, nasal aerosol, ointment, cream, semi-solid, and/or suspension. In some embodiments, nasal formulations according to the present disclosure can be a drop delivery formulation. In some embodiments, nasal formulations can be a spray or atomizer delivery formulation.

In other embodiments, pharmaceutically acceptable compositions of this disclosure can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutical compositions herein for inhalation and/or insufflation can include solutions and suspensions in pharmaceutically acceptable, aqueous or organic solvents, or mixtures thereof, and powders. In some embodiments, liquid or solid pharmaceutical compositions herein can contain one or more suitable pharmaceutically acceptable excipients as set out above. In some embodiments, pharmaceutical compositions herein can be administered by an oral or a nasal respiratory route for local or systemic effect.

In some embodiments, pharmaceutical compositions and formulations herein can be suitable for intranasal administration or inhalation, such as delivered in the form of a dry powder inhaler, liquid inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In some embodiments, having a pressurized aerosol, a dosage unit can be determined by providing a valve to deliver a metered amount. In some embodiments, a pressurized container, pump, spray or nebulizer can contain a solution or suspension of the active compound (e.g., latanoprost, bimatoprost, tafluprost) using a mixture of ethanol and a propellant as a solvent, which can additionally contain a lubricant. In some embodiments, capsules and cartridges (made, for example, from gelatin) for use in an inhaler or insufflator herein can be formulated to contain a powder mix of the active compound (e.g., latanoprost, bimatoprost, tafluprost) and a suitable powder base such as lactose or starch. In some embodiments, formulations herein can be presented in unit-dose or multi-dose containers, for example sealed ampoules or vials, and can be stored in a freeze-dried (lyophilized) condition requiring only the addition of the sterile liquid carrier immediately prior to use.

In some embodiments, formulations disclosed herein can be about 1.1, about 1.2, about 1.3, about 1.4, about 1.5, about 2.0, about 2.5, about 3.0 or more times the viscosity compared to an eye formulation containing latanoprost, or other prostaglandin F2 alpha agonist (e.g. vyzulta, bimatoprost, tafluprost) or a combination containing latanoprost. In some embodiments, an increased viscosity contemplated for formulations herein can be used to increase adherence to nasal mucosa and/or to increase persistence of exposure to the agents (e.g., latanoprost) contemplated herein.

In some embodiments, formulations disclosed herein in sterile pharmaceutically acceptable solvents can be nebulized by use of gases. In some embodiments, nebulized solutions herein can be breathed directly from the nebulizing device or the nebulizing device can be attached to a face mask, tent or intermittent positive pressure breathing machine. In some embodiments, solution, suspension, emulsion or powder compositions herein can be administered nasally, from devices which deliver the composition in an appropriate dosage.

Conventional methods, known to those of ordinary skill in the art of medicine, can be used to administer a pharmaceutical composition herein to a subject, depending upon the type of disease to be treated or the site of the disease. In some embodiments, pharmaceutical compositions herein can also be administered via other conventional routes, e.g., administered orally, parenterally, by inhalation spray, topically, rectally, nasally, buccally, vaginally or via an implanted reservoir.

In some embodiments, formulations herein for topical (e.g., transdermal or transmucosal) administration can penetrant appropriate to the barrier to be permeated. In some embodiments, transmucosal administration of formulations herein can be accomplished through the use of nasal sprays, aerosol sprays, tablets, or suppositories, and transdermal administration can be via ointments, salves, gels, patches, or creams as generally known in the art.

The term “intranasal(ly),” as used herein can refer to application of formulations of the disclosure to a surface on the skin, mucosa and/or tissues of nasal passages, e.g., nasal mucosa, sinus cavity, nasal turbinates, or other tissues and lining nasal passageways. In some embodiments, intranasal administration can include administration via the nose, either with or without concomitant inhalation during administration. In some embodiments, intranasal administration of a formulation herein can occur through contact of the formulation herein with nasal mucosa, nasal turbinates and/or sinus cavity. In some embodiments, inhalation administration of a formulation herein can include intranasal administration or can include oral inhalation. In some embodiments, administration of formulation herein can also include, but is not limited to, contact with the oral mucosa, bronchial mucosa, sinus passageway mucosa and other epithelia. Non-limiting examples of administration can include, but is not limited to, endosinusial, endotracheal, transtracheal, intratracheal, intrabronchial, intracavernous, intrapleural, intrapulmonary, intrasinal, nasal, oral, parenteral, inhalation, subcutaneous, submucosal, mucosal, and transmucosal.

Formulations according to the present disclosure can be administered in an aqueous solution as a nasal or pulmonary spray and can be dispensed in spray form by a variety of methods known to those skilled in the art. In some embodiments, the formulations herein can be presented in multi-dose containers, for example in a sealed dispensing system. Additional aerosol delivery forms can include, but are not limited to, compressed air-, jet-, ultrasonic-, and piezoelectric-nebulizers, which deliver the biologically active agent dissolved or suspended in a pharmaceutical solvent, e.g., water, ethanol, or a mixture thereof.

In some embodiments, methods of administering a formulation disclosed herein can include administering the formulation intranasally by topical, spray, mist, drop, or aerosol delivery of the formulation.

In some embodiments, the present disclosure provides methods for reducing intracranial pressure in the subject. In some embodiments, the present disclosure provides methods for increasing CSF outflow in a subject. In other embodiments, a subject to be treated by the methods herein can be a mammal. Mammals include, but are not limited to, farm animals such as livestock, sport animals, pets, primates, horses, dogs, cats, mice and rats. In some embodiments, a subject to be treated by the methods described herein can be a human subject. In some embodiments, human subjects can be adult, young adult, adolescent, a child, an infant or an unborn fetus.

In certain embodiments, a subject can have, be at risk for, or be suspected of having, Spaceflight-Associated Neuro-Ocular Syndrome, idiopathic intracranial hypertension, pseudotumor cerebri syndrome, or the like. In some embodiments, a subject having or suspected of having Spaceflight-Associated Neuro-Ocular Syndrome, idiopathic intracranial hypertension, pseudotumor cerebri syndrome, or the like can be identified by routine medical examination. In other embodiments, a subject to be treated by the methods described herein can have optic disc swelling, choroidal engorgement, positional headache, pulse-synchronous tinnitus, cerebral venous sinus stenosis, refractive shifts, or a combination thereof.

In certain embodiments, defects in CSF outflow in a subject can result in elevated intracranial pressure ICP and other side effects. In some embodiments, astronauts can experience increased levels of ICP due to the duration of time in space flight which can cause side effects such as optic deficiencies. Current procedures for reducing ICP include invasive surgery, weight loss, and medications that block the production of CSF (e.g., inflow) which can have other side effects. In some embodiments, a pharmaceutical composition for delivery to a subject suffering from such a condition is provided herein to ensure decrease of ICP in the subject by influencing the outflow of CSF both in space flight and on Earth.

In some embodiments, compositions that block the production of CSF (e.g., an inflow of CSF) have been established for reducing ICP. When applied, however, the decrease in inflow of CSF (e.g., production) was not efficient in decreasing ICP because blocking the production of CSF can lead to a shift in the cephalad fluid in the intracranial space thereby increasing ICP. In some embodiments, blocking the production of CSF can impair CSF reabsorption. In some embodiments, a universal directed pharmaceutical composition for intranasal administration described herein can induce outflow of CSF and/or reduce ICP while avoiding one or more side effects of current treatments. In accordance with this understanding, nasal formulations disclosed herein serve such a purpose while reducing side effects.

In some embodiments, nasal formulations disclosed herein can be used for increased ICP in astronauts (e.g., individuals exposed to microgravity for increased durations). In some embodiments, the disclosed nasal formulations can be used to treat astronauts. In accordance with these embodiments, over 50% of long-duration spaceflight astronauts have been observed to develop optic disc swelling, choroidal engorgement, and refractive shifts that comprise the condition known as SANS. Persistent optic disc edema can lead to axonal death, resulting in permanent peripheral and/or central vision loss. Optic disc swelling, as measured by retinal nerve fiber layer thickness on optic coherence tomography and by fundus photography, can be detectable within 90 days of exposure to microgravity. In some cases, there is radiographic evidence for cerebral edema and ventricular enlargement that can be seen by brain MRI performed shortly after return to Earth.

In some embodiments, formulations disclosed herein can be used to reduce the risk or treat SANS. In certain embodiments, formulations herein can be used to identify preventative and treatment countermeasures to mitigate changes in ocular structure and function and ICP during spaceflight. Noninvasive measures can be used to monitor the intraocular pressure (TOP) of the subject and ICP response. At least one advantage to the methods described herein includes use of Food and Drug Administration (FDA) approved agents (e.g., latanoprost) that are safe and tolerable with fewer side effects. In some embodiments, upon implementation, formulations described and used herein can decrease the time for each astronaut to comply with a treatment. In some embodiments, formulations herein do not adversely affect circadian rhythm, exercise tolerance, or cognition. In some embodiments, formulations herein that can be of use to treat or prevent SANS can be easily altered or ceased without significant complications such as those known for other treatments such as surgical invasion. In some embodiments, formulations herein can be used as a countermeasure for astronauts that experience long duration spaceflight and exploration missions.

Compositions disclosed herein are contemplated of use in other conditions. For example, in some embodiments, cephalad fluid shifts leading to increased venous vascular volume, impaired CSF reabsorption, and/or increased ICP can drive negative effects of edema formation. In other embodiments, other contributory factors can include genetic variants in 1-carbon metabolism, increased pCO2 in the spacecraft environment, valsalva maneuvers during resistive exercise, and bony optic canal diameter. Current treatments for elevated ICP, include pharmacotherapy with diuretics, surgical release of CSF into adjacent or distant body spaces, and cerebral venous sinus stenting of obstructive lesions; however these treatments are not conducive to individuals experiencing microgravity or can increase the risk for worsening of existing health hazards (e.g. bone loss or kidney stone formation). In some embodiments, nasal formulations disclosed herein can be used to reduce the risk of onset, treat or prevent these conditions and provide alternative treatments for subjects suffering from these conditions such as, but not limited to, an individual subjected to short-term or long-term space travel.

As used herein, “an effective amount” refers to the amount of nasal formulation herein that can confer a therapeutic effect on the subject, either alone or in combination with one or more other active agents. Determination of whether an amount of the nasal formulation disclosed herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. Generally, a maximum dose of the individual components or combinations thereof is that which can be used, that is, to the highest safe dose according to sound medical judgment.

Empirical considerations, such as the half-life, generally will contribute to the determination of the dosage. Frequency of administration can be determined and adjusted over the course of therapy, and is generally, but not necessarily, based on treatment and/or suppression and/or amelioration and/or delay of a target disease/disorder. Alternatively, sustained continuous release nasal formulations can be appropriate. Various formulations and devices for achieving sustained release are known in the art.

In some embodiments, dosages of nasal formulations as described herein can be determined empirically for a subject to receive one or more doses of the nasal formulations herein of the same or different matters. In other embodiments, administration of any of the nasal formulations described herein can be about 0.01 mg/ml to about 20.0 mg/ml or more depending on individual factors. For repeated administrations over several days or longer, depending on the condition, the treatment can be sustained until a desired suppression of symptoms occurs or until sufficient therapeutic levels are achieved to alleviate a target disease or disorder, or a symptom thereof. In some embodiments, dosage regimens can be used to treat a subject from about one to seven times a day or a week or other regimen is contemplated. In some embodiments, dosing ranging from about 0.01 mg/ml to about 20.0 mg/ml active agent (e.g., latanoprost) per day or multiple times per day can be used. In some embodiments, dosing frequency can be three times a day or more, two times a day, once every day, every other day, twice a week, once week, every 2 weeks, every 4 weeks, every 5 weeks, every 6 weeks, every 7 weeks, every 8 weeks, every 9 weeks, or every 10 weeks; or once every month, every 2 months, or every 3 months, or longer or whenever ICP increases and/or CSF outflow decreases. The progress of this therapy can be easily monitored by conventional techniques and assays. The dosing regimen can vary over time.

In some embodiments, dosage of a nasal formulation as described herein can depend on the type and severity of the disease/disorder, whether the formulation is administered for preventive or therapeutic purposes, previous therapy, the subject's clinical history and response to the formulation, and the discretion of the attending physician. In other embodiments, a nasal formulation herein can be administered until a dosage is reached that achieves a desired result. In certain embodiments, a desired result can be a decrease in the intracranial pressure in the subject. In some embodiments, a desired result can be about 1% to about 100% decrease or about 10% to about 20% to about 30% or more, to about 100% decrease in intracranial pressure in a subject after administration of a nasal formulation disclosed herein compared to a subject not receiving such a treatment. In some embodiments, a desired result can be an increase in the cerebrospinal fluid (CSF) outflow in a subject. In some embodiments, a desired result can be about 1% to about 100% or about 10%, about 20%, about 30% or more to about 100% increase in CSF outflow in a subject after administration of a nasal formulation disclosed herein. Methods of determining whether a dosage resulted in the desired result would be evident to one of skill in the art. In some embodiments, administration of nasal formulations herein can be continuous or intermittent, depending, for example, upon the subject's physiological condition, whether the purpose of the administration is therapeutic or prophylactic, and other factors known to skilled practitioners. In some embodiments, administration of nasal formulations herein can be continuous over a preselected period of time or can be in a series of dosage treatments.

In some embodiments, methods of treating a subject can include the step of administering formulations disclosed herein intranasally to a subject in need thereof. In some examples, a formulation can be administered to a subject via nasal spray, a metering, atomizing spray pump. Each actuation of the pump delivers a single dosage of the drug substance to the subject.

Compositions of the present invention can, if desired, be presented in a pack or dispenser device, such as an FDA approved kit, which can contain one or more-unit dosage forms containing the active ingredient. The pack can, for example, comprise metal or plastic foil, such as a blister pack. The pack or dispenser device can be accompanied by instructions for administration. The pack or dispenser can also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, can be of labeling approved by the U.S. Food and Drug Administration for prescription drugs or of an approved product insert. Kit compositions comprising a preparation of a formulation disclosed herein containing for example, latanoprost, or other prostaglandin F2 alpha agonist (e.g. bimatoprost and/or tafluprost and/or vyzulta) in a compatible pharmaceutical carrier can also be prepared, placed in an appropriate container, and labeled for treatment of an indicated condition, as disclosed herein.

In some embodiments, kits containing nasal formulations disclosed herein including at least latanoprost and at least one container are contemplated. In other embodiments, kits can include instructions for use in accordance with any of the methods described herein. The included instructions can comprise a description of administration of the pharmaceutical composition for delivering the therapeutic agent or diagnostic agent encapsulated therein or for treating Spaceflight-Associated Neuro-Ocular Syndrome, idiopathic intracranial hypertension. pseudotumor cerebri syndrome, or the like according to any of the methods described herein. The kit can further include a description of selecting an individual suitable for treatment based on identifying whether that individual has, is suspected of having, or is at risk for Spaceflight-Associated Neuro-Ocular Syndrome, idiopathic intracranial hypertension. pseudotumor cerebri syndrome, or the like.

In some embodiments, instructions relating to the use of the nasal formulations described herein, generally include information as to dosage, dosing schedule, and route of administration for the intended treatment. The containers can be unit doses, bulk packages (e.g., multi-dose packages) or sub-unit doses. Instructions supplied in the kits disclosed herein can be written instructions on a label or package insert (e.g., a paper sheet included in the kit), but machine-readable instructions (e.g., instructions carried on a magnetic or optical storage disk) can also be acceptable. In some embodiments, kits as described herein can be in suitable packaging. Suitable packaging includes, but is not limited to, vials, bottles, jars, flexible packaging (e.g., sealed Mylar or plastic bags), and the like. Also contemplated herein are packages for use in combination with a specific device, such as an inhaler, nasal administration device (e.g., an atomizer) or any suitable device for nasal delivery.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

In one exemplary method, anatomic delivery of a fluorescent tracer (AlexaFluor⁶⁴⁷-ovalbumin solution) into the lateral ventricle was assessed using an animal model. In this example, animals were administered fluorescent-labelled ovalbumin by stereotaxic injection into the right lateral ventricle. Injected animals were then sacrificed, and brain tissue was harvested 60 minutes after injection. The 60-minute timepoint for tissue harvest was selected based on increased recovery observed at 60 minutes compared to 30 minutes. Harvesting at a later timepoint was not performed to avoid potential contamination of specimens by hematogenous CSF absorption of fluorescent tracer and recirculation throughout the tissues via arterial flow. Other tissues (spleen, liver) from injected animals did not demonstrate significant levels of fluorescent tracer relative to uninjected animals after 60 minutes.

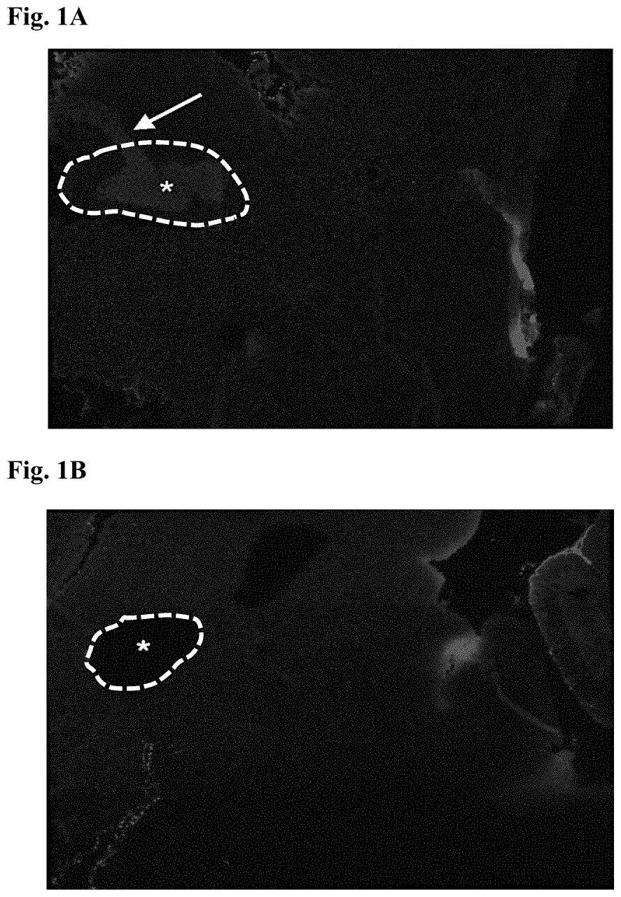

FIG. 1A illustrates a representative photomicrograph image of a brain section at 20× magnification prepared from brain tissue harvested from a mouse after stereotaxic injection of fluorescent-labelled ovalbumin into the right lateral ventricle. In the injected mouse, the fluorescent-labelled ovalbumin was localized within the ventricle (see area within dashed line of FIG. 1A, marked with an asterisk) and along the injection track (see arrow in FIG. 1A). FIG. 1B shows a representative photomicrograph image of a brain section at 20× magnification prepared from brain tissue harvested from a control, uninjected mouse wherein there is no fluorescence detected in the ventricle (see area within dashed line of FIG. 1B, marked with an asterisk).

### Example 2

In another exemplary method, compositions including prostaglandin F2-alpha analogues were used to analyze increased lymphatic channel contractility in the nasal mucosa of experimental animals. In this example, prostaglandin F2-alpha analogues latanoprost, bimatoprost, travoprost, latanoprostene bunod, and tafluprost were administered via an intranasal vehicle into the nasal mucosa of mice. The chemical structures of the prostaglandin F2-alpha analogues tested are provided in Table 1.

TABLE 1

| Prostaglandin F2-alpha Analogue | Prostaglandin F2-alpha Analogue |
|---|---|
| Name | Chemical Structure |
| Bimatoprost | 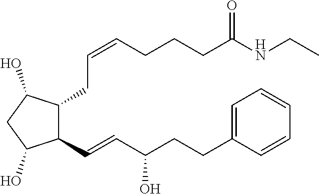 [SMILES: CCNC(=O)CCC/C=C\C[C@@H]1[C@@H](/C=C/[C@@H](O)CCc2ccccc2)[C@H](O)C[C@@H]1O] |
| Latanoprost | 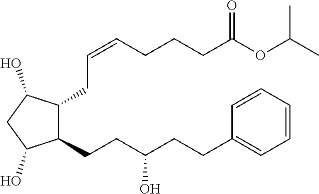 [SMILES: CC(C)OC(=O)CCC/C=C\C[C@@H]1[C@@H](CC[C@@H](O)CCc2ccccc2)[C@H](O)C[C@@H]1O] |
| Latanoprost bunod | 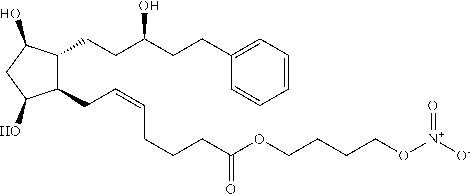 [SMILES: O=C(CCC/C=C\C[C@@H]1[C@@H](CC[C@@H](O)CCc2ccccc2)[C@H](O)C[C@@H]1O)OCCCCO[N+](=O)[O-]] |
| Tafluprost | 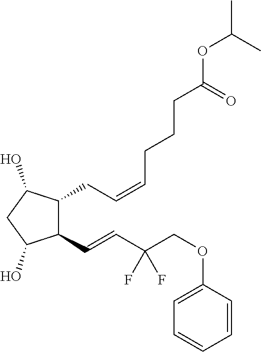 [SMILES: CC(C)OC(=O)CCC/C=C\C[C@@H]1[C@@H](/C=C/C(F)(F)COc2ccccc2)[C@H](O)C[C@@H]1O] |
| Travoprost | 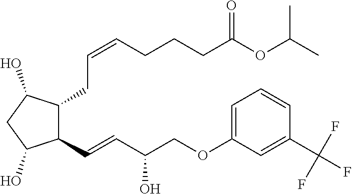 [SMILES: CC(C)OC(=O)CCC/C=C\C[C@@H]1[C@@H](/C=C/[C@@H](O)COc2cccc(C(F)(F)F)c2)[C@H](O)C[C@@H]1O] |

In this example, a total of 67 animals were studied using a scaled dosing technique in which each prostaglandin analog was evaluated at its commercial ophthalmic concentration as well as one or two more concentrated forms (either 5× or 10×).

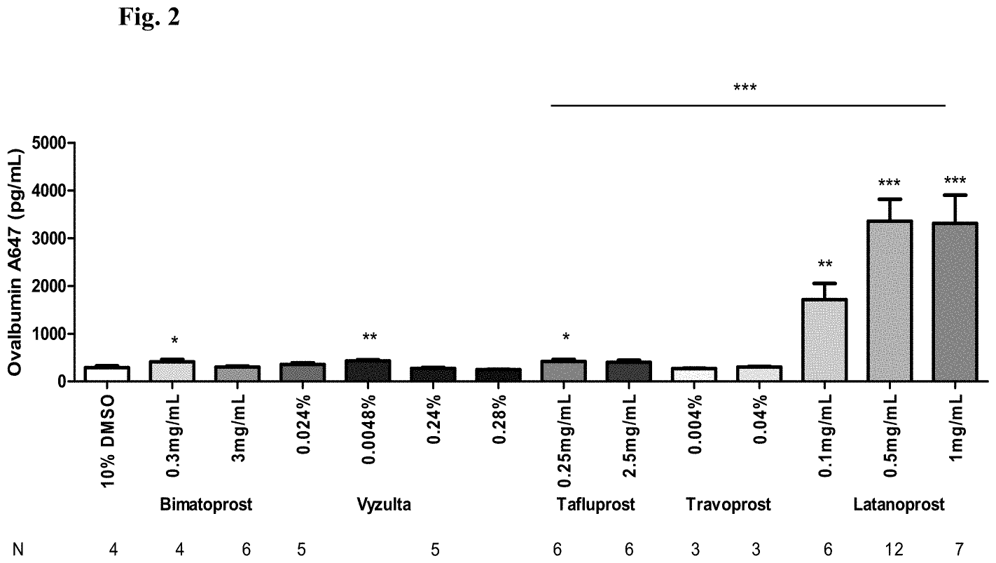

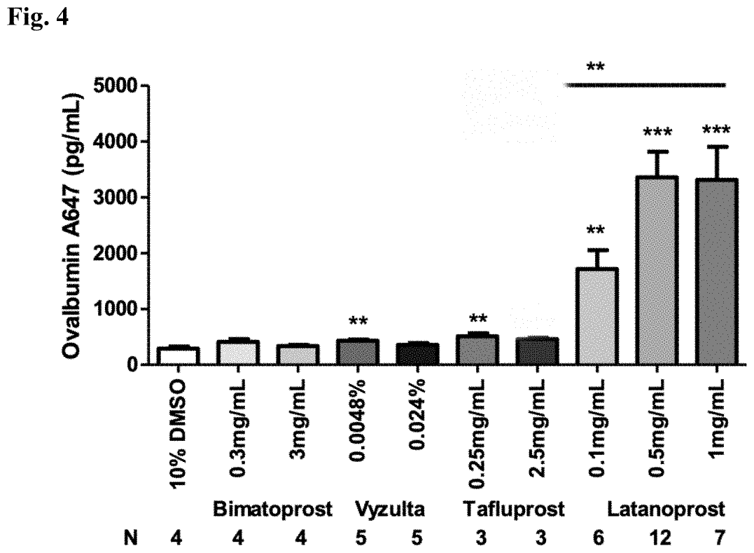

In this exemplary method, intranasal delivery of latanoprost resulted in a semi dose-dependent increase in tracer recovery from the nasal mucosa after 60 minutes, with approximately 10-fold increase relative to control animals when 0.5 mg/ml or 1 mg/ml solution was used (p<0.001 compared to control) (FIGS. 2-4). The standard ophthalmic dose of latanoprost, 0.1 mg/ml, resulted in approximately 6-fold increased tracer recovery (p<0.01 vs control) (FIGS. 2-4). The remaining prostaglandin analogs demonstrated lesser increases in recovery of fluorescent tracer from the nasal mucosa. Commercial concentrations of bimatoprost and tafluprost demonstrated an approximately 1.5-fold increase in nasal tracer recovery relative to vehicle-treated controls (p<0.05 vs control); however, nasal tracer recovery did not appear to improve at higher doses (FIGS. 2-4). Neither travoprost nor latanoprostene bunod demonstrated a measurable effect at any tested dose (FIGS. 2-4). Additionally, use of concentrated latanoprost (e.g. 0.5 mg/ml and 1 mg/ml) demonstrated a significant increase in tracer recovery over all tested prostaglandin analogs and 0.1 mg/ml latanoprost (FIGS. 2-4).

Regarding FIG. 2, a comparison of the effect of latanoprost intranasal application to other PG F2alpha analogues in the recovery from nasal turbinates of fluorescently tagged ovalbumin after CSF injection after single dose application of each drug was evaluated. All statistical comparisons are relative to controls except as indicated by the horizontal bar comparing 1 mg/ml latanoprost with 0.25 mg/ml tafluprost; *=p<0.05, **=p<0.01, ***=p<0.001.

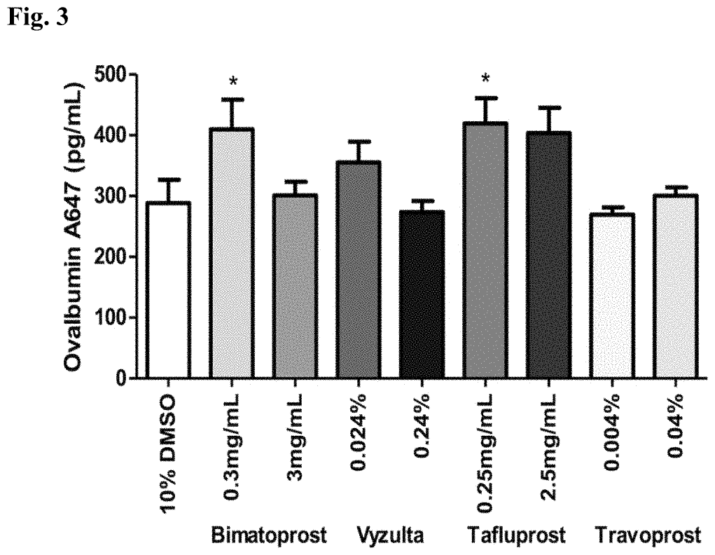

In FIG. 3, it was demonstrated by fluorometric analysis of nasal turbinate homogenates after prostaglandin F2alpha agonist bimatoprost, latanoprostene bunod, tafluprost and travoprost inhalation and ventricular tracer injection. Some enhanced tracer recovery relative to controls was seen with the lower doses of bimatoprost and tafluprost. Error bars denote SEM. Asterisks indicate p<0.05 relative to controls.

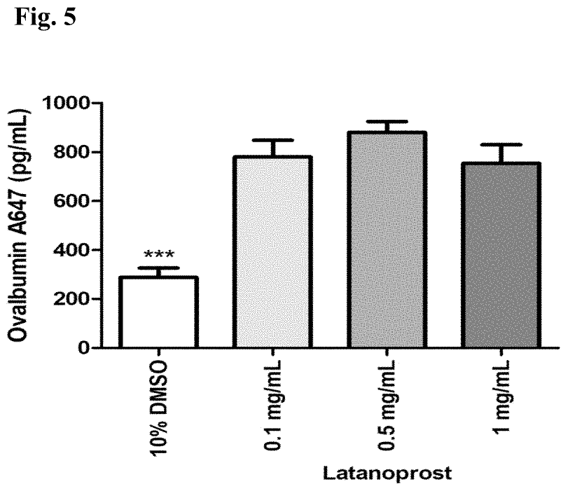

In another example, an additional 14 animals were administered inhaled doses of latanoprost (either 0.1, 0.5, or 1 mg/ml) once a day for three (3) consecutive days. Nasal turbinates were isolated 1 hour after ventricular tracer injection and between 7 and 9 hours after the final latanoprost dose was given intranasally according to the exemplary methods described herein. A nearly 3-fold increase in tracer recovery relative to controls (p<0.05) was observed, with no statistically significant difference observed amongst the 3 doses (FIG. 5). In this example, latanoprost, when given in daily doses for 3 days, showed a sustained ability to increase tracer recovery even several hours after the last dose was administered.

Accordingly, latanoprost demonstrated a dose-dependent ability to increase recovery of a CSF-based fluorescent tracer substance from the nasal mucosa of experimental animals shortly after intranasal inhalation. This effect may be a result of increased lymphatic channel contractility mediated by latanoprost was not be demonstrated by related prostaglandin F2-alpha analogues tested.

### Exemplary Methods

Commercially available prostaglandin F2-alpha analogues used in the treatment of glaucoma were selected for evaluation; these included latanoprost, bimatoprost, travoprost, latanoprostene bunod, and tafluprost. Initial experiments were conducted with topical ophthalmic preparations, and pharmaceutical grade drug also was obtained and placed into solution for use in a more concentrated form. Agents were solubilized in DMSO and diluted in normal saline.

For single inhalation trails described herein, awake adult male and female C57/BL6 mice (approx. 6 weeks of age) were administered intranasal vehicle (10% DMSO) or drug solution (total 8 μl) by placing droplets (1-2 μl) of liquid at the nares using a micropipette and allowing the animal to inhale the liquid spontaneously. Anesthesia with ketamine and xylazine was administered via intraperitoneal injection, and anesthetized animals were placed within a stereotaxic apparatus. For multiday studies, intranasal drug administration was performed on awake animals daily using the same procedure described above. Approximately 6 hours after the third daily dose was given, animals were anesthetized and placed within a stereotaxic apparatus. A midline incision was created along the scalp, and the right lateral ventricle was targeted with placement of a 26-gauge needle to a depth of 3 mm into the brain at a position 1 mm lateral and 0.3 mm anterior to bregma. A 5 μl intraventricular injection of fluorescent tracer (AlexaFluor⁶⁴⁷-ovalbumin solution) was delivered at a rate of 100 nl/seconds using a Hamilton syringe and an electric pump. Animals remained anesthetized for the 1 hour recovery period post injection and were then euthanized with a lethal overdose of ketamine followed by secondary cervical dislocation. Nasal turbinates were harvested, and tissue extracts were analyzed by ELISA.

The experimental technique was validated by evaluating recovery of fluorescent tracer from nasal mucosa after ventricular injection in the absence of intranasal treatment. Time points for tissue harvest were selected based on increased recovery at 60 minutes versus 30 minutes, with later harvest raising concern for potential contamination of specimens by hematogenous CSF absorption and recirculation of tracer throughout the tissues via arterial flow.

Statistical analyses were conducted with Stata using either two-tailed Student's t-tests or ANOVA RM with Dunnet's post hoc analysis.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A nasal formulation comprising,
at least one agent comprising latanoprost, bimatoprost, tafluprost, vyzulta or pharmaceutically acceptable salt thereof or combination thereof; at least one adherence agent for prolonging nasal mucosal interaction of the formulation; and at least one pharmaceutically acceptable carrier or diluent.

2. The nasal formulation according to claim 1, wherein the carrier or diluent is selected from solid carriers or diluents, liquid carriers or diluents, gel carriers or diluents or a combination thereof.

3. The nasal formulation according to claim 1, wherein the at least one adherence agent comprises at least one of cellulose or a derivative thereof, a starch, a wax, a gel, a synthetic polymer, and a natural polymer.

4. The nasal formulation according to claim 2, wherein the carrier or diluent is a liquid carrier or diluent comprising at least one of water, propylene glycol and pharmaceutically acceptable alcohols.

5. The nasal formulation according to claim 1, wherein the agent comprises latanoprost comprising a concentration of about 0.01 mg/ml to about 20.0 mg/ml.

6. The nasal formulation according to claim 1, wherein the agent comprises latanoprost and the latanoprost comprises a viscous liquid or semi-solid particles.

7. The nasal formulation according to claim 1, wherein the formulation comprises a solution, a suspension, or an emulsion.

8. The nasal formulation according to claim 1, wherein the nasal formulation further comprises at least one agent of carbopol, chitosan, DMSO (dimethyl sulfoxide), sodium carboxymethyl cellulose (NaCMC), hydroxypropyl methylcellulose (HPMC), hydroxypropyl cellulose methylcellulose, poloxamer, polyoxyethylene, pluronic-poly(acrylic acid) copolymer, carbomer, chitosan, polyvinyl alcohol (PVA), poly(N-isopropylacrylamide) (PNiPAAm), methocel A4M, polymethacrylic acid and polyethylene glycol (P(MAA-g-EG), and polyvinylacetal diethylamino acetate.

9. The nasal formulation according to claim 1, wherein the pharmaceutical formulation comprises a drop delivery formulation for nasal administration.

10. The nasal formulation according to claim 1, wherein the pharmaceutical formulation comprises a spray or atomizer delivery formulation.

11. The nasal formulation according to claim 1, wherein the nasal formulation comprises a formulation comprising higher viscosity than that of an eye formulation.

12. A method of increasing cerebrospinal fluid (CSF) outflow in a subject in need thereof, the method comprising administering the nasal formulation according to claim 1 to the subject.

13. The method of increasing CSF outflow according to claim 12, wherein latanoprost or a pharmaceutically acceptable salt thereof comprises a concentration of about 0.01 mg/ml to about 20.0 mg/ml.

14. A method of reducing intracranial pressure (ICP) in a subject, the method comprising:
administering a nasal formulation according to claim 1 to the subject, wherein administration of the nasal formulation reduces intracranial pressure in the subject.

15. The method according to claim 14, wherein the subject comprises a subject having at least one of optic disc swelling, choroidal engorgement, positional headache, pulse-synchronous tinnitus, cerebral venous sinus stenosis, and refractive shifts.

16. The method according to claim 14, wherein the subject comprises a subject having at least one of Spaceflight-Associated Neuro-Ocular Syndrome, idiopathic intracranial hypertension, and pseudotumor cerebri syndrome.

17. The method according to claim 12, wherein administering the nasal formulation to the subject comprises intranasally, by topical, spray, mist, drop, or aerosol delivery of the nasal formulation to the subject.

18. The method according to claim 12, wherein administering the nasal formulation to the subject comprises intranasally, by topical, spray, mist, drop, or aerosol delivery of the nasal formulation to the subject at least once a day.

19. A kit comprising:
(a) nasal formulation according to claim 1; and
(b) at least one container.

20. The kit according to claim 19, wherein the kit is of use to increase cerebrospinal fluid (CSF) outflow in a subject in need thereof.

21-22. (canceled)
